(19) United States
(12) Patent Application Publication
(10) Pub. No.: US 2025/0137981 A1
(43) Pub. Date: May 1, 2025

(54) SYSTEMS AND METHODS OF MEASURING SULFUR COMPOSITION IN A GAS AND LIQUIFIED GAS

(71) Applicant: Saudi Arabian Oil Company, Dhahran (SA)

(72) Inventors: Frederick M. Adam, Dhahran (SA); Guillaume Robert Jean-Francois Raynel, Dhahran (SA); Mohammed Sharedah, Al Ahsa (SA); Shabbir Ghulam, Dhahran Hills (SA)

(21) Appl. No.: 18/495,421

(22) Filed: Oct. 26, 2023

(51) Int. Cl. G01N 33/00; C07C 22/04

(52) U.S. Cl. CPC G01N 33/0013; C07C 22/04; C07C 2602/10

(57) ABSTRACT

This disclosure relates to methods of determining the sulfur-content in a gas sample or liquefied petroleum gas sample comprising sulfur-containing compounds, including derivatizing the sulfur-containing compounds and analyzing the derivatized sulfur-containing compounds.

### TECHNICAL FIELD

This document relates to methods of determining the sulfur-content in a gas sample or liquefied petroleum gas sample including sulfur-containing compounds, including derivatizing the sulfur-containing compounds and analyzing the derivatized sulfur-containing compounds.

### BACKGROUND

To meet safety, environmental, and product standards, it is necessary to accurately characterize the sulfur-containing compounds within natural gas samples. Conventional methods of characterization, such as the use of a sampling valve and gas chromatograph equipped with a selective detector, suffer from a number of drawbacks. First, a gas or liquid natural gas sample is collected and often transported for analysis. However, in addition to the safety risks associated with the transport of pressurized gas samples (such as gas leaks and subsequent hazards), sulfur species may absorb on the walls of the storage containers affecting the analytical data. Second, the impurities within gas and liquefied petroleum gas sample often damage analytical systems, such as gas chromatography columns, resulting in costly maintenance and inaccurate measurements. Lastly, conventional methods require comparison to analytical standards, which are difficult to source and have short shelf-lives.

Therefore, there is a growing need to develop new methods of characterizing the sulfur-containing compounds in a gas sample or liquefied petroleum gas sample that are safer and more accurate than existing methods.

### SUMMARY

Provided in the present disclosure are methods of determining the sulfur-content in a gas sample or liquefied petroleum gas sample comprising one or more sulfur-containing compounds, the method comprising:
contacting the gas sample or liquefied petroleum gas sample comprising one or more sulfur-containing compounds with a derivatization agent to form one or more derivatized sulfur-containing compounds; and
analyzing the gas sample or liquefied petroleum gas sample comprising one or more derivatized sulfur-containing compounds to determine the sulfur-content;
wherein the method determines the total concentration of the one or more sulfur-containing compounds in the gas sample or liquefied petroleum gas sample, wherein the one or more sulfur-containing compounds are selected from hydrogen sulfide, carbonyl sulfide, carbon disulfide, mercaptans, dialkyl sulfides, and combinations thereof.

In some embodiments, the gas sample or liquefied petroleum gas sample is a gas sample.

In some embodiments, the gas sample or liquefied petroleum gas sample is a liquefied petroleum gas sample.

In some embodiments, the gas sample or liquefied petroleum gas sample comprises hydrogen sulfide, carbonyl sulfide, carbon disulfide, mercaptans, and dialkyl sulfides.

In some embodiments, the gas sample or liquefied petroleum gas sample comprises hydrogen sulfide.

In some embodiments, the gas sample or liquefied petroleum gas sample comprises dialkyl sulfides.

In some embodiments, the derivatization agent is of formula (I):

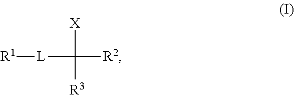

wherein:
X is halo or tosyl;
L is selected from bond, C₁₋₁₂ alkyl, C₂₋₁₂ alkenyl, C₂₋₁₂ alkynyl, and —C(O)—, wherein the C₁₋₁₂ alkyl, C₂₋₆ alkenyl, and C₂₋₆ alkynyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R¹ is selected from C₆₋₁₀ aryl and 5-14 membered heteroaryl, wherein the C₆₋₁₀ aryl or 5-14 membered heteroaryl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a}; R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl.

In some embodiments, the derivatization agent is of formula (II):

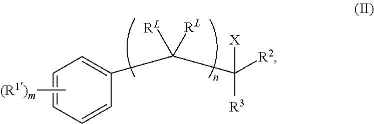

wherein:
X is halo or tosyl;
each R^{L} is independently selected from H, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
each R^{1′} is independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl;
m is an integer selected from 0 to 5; and
n is an integer selected from 0 to 12.

In some embodiments, the derivatization agent is of formula (III-A) or (III-B):

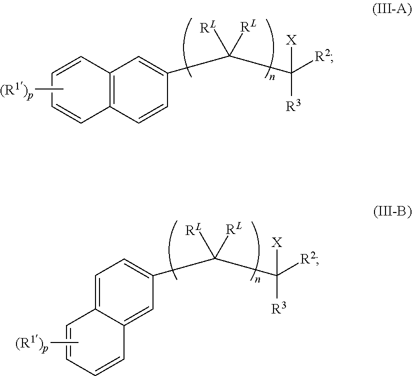

wherein:
X is halo or tosyl;
each R^{L} is independently selected from H, C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
each R¹ is independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl;
n is an integer selected from 0 to 12; and
p is an integer selected from 0 to 6.

In some embodiments, the derivatization agent is selected from benzyl chloride, benzyl bromide, benzyl iodide, 1-(chloromethyl)naphthalene, 1-(bromomethyl)naphthalene, 1-(iodomethyl)naphthalene, 2-(chloromethyl)naphthalene, 2-(bromomethyl)naphthalene, and 2-(iodomethyl)naphthalene.

In some embodiments, the derivatization agent is benzyl bromide.

In some embodiments, contacting the gas sample or liquefied petroleum gas sample comprising one or more sulfur-containing compounds with a derivatization agent to form one or more derivatized sulfur-containing compounds occurs at room temperature.

In some embodiments, one or more of the derivatized sulfur-containing compounds are soluble in the derivatization agent.

In some embodiments, one or more of the derivatized sulfur-containing compounds are insoluble in the derivatization agent.

In some embodiments, one or more of the derivatized sulfur-containing compounds are salts.

In some embodiments, analyzing the gas sample or liquefied petroleum gas sample comprises a first analysis step, wherein:
the first analysis step comprises analyzing the one or more derivatized sulfur-containing compounds that are soluble in the derivatization agent.

In some embodiments, analyzing the gas sample or liquefied petroleum gas sample comprises a first analysis step, wherein:
the second analysis step comprises treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.

In some embodiments, treating the one or more derivatized sulfur species that are insoluble in the derivatization agent is a thermal treatment process or a chemical treatment process.

In some embodiments, the method determines the identity of the sulfur-containing compounds.

In some embodiments, the method determines the concentration of each of the sulfur-containing compounds.

In some embodiments, the derivatization agent does not react with hydrocarbons present in the gas sample or liquefied petroleum gas sample, wherein the hydrocarbons are selected from methane, ethane, propane, butane, pentane, hexane, heptane, octane, benzene, toluene, xylenes, and combinations thereof.

Provided in the present disclosure are methods of determining the sulfur-content in a gas sample or liquefied petroleum gas sample comprising one or more sulfur-containing compounds, the method comprising:
contacting the gas sample or liquefied petroleum gas sample comprising one or more sulfur-containing compounds with a derivatization agent to form one or more derivatized sulfur-containing compounds;
performing a first analysis step comprising analyzing the one or more derivatized sulfur-containing compounds that are soluble in the derivatization agent; and
performing a second analysis step comprising treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.

### DESCRIPTION OF DRAWINGS

FIG. 1 shows an exemplary scheme of the reactions of water and sulfide with a derivatization agent, such as a benzyl halide, to form a benzyl alcohol and benzyl thiol, respectively. “Aromatic” refers to an aryl or heteroaryl ring; “H” refers to hydrogen; “Ar” refers to an aryl or heteroaryl ring; and “Alk” refers to an alkyl group.

FIG. 2 shows an exemplary scheme of the reaction of a mercaptan with a derivatization agent, such as a benzyl halide, to form a benzyl alkyl sulfide. “Aromatic” refers to an aryl or heteroaryl ring; “H” refers to hydrogen; “Ar” refers to an aryl or heteroaryl ring; “Alk” refers to an alkyl group; “Me” refers to methyl; “Et” refers to ethyl; and “Pr” refers to propyl.

FIG. 3 shows an exemplary scheme of the reaction of a dialkyl sulfide with a derivatization agent, such as a benzyl halide, to form a sulfonium salt precipitate. “Aromatic” refers to an aryl or heteroaryl ring; “H” refers to hydrogen; “Ar” refers to an aryl or heteroaryl ring; and “Alk” refers to an alkyl group.

FIG. 4 shows an exemplary scheme of the reactions of carbonyl sulfide and carbonyl disulfide with a derivatization agent, such as a benzyl halide, to form a xanthate and trithiocarbonate, respectively. “Aromatic” refers to an aryl or heteroaryl ring; “H” refers to hydrogen; “Ar” refers to an aryl or heteroaryl ring; and “Alk” refers to an alkyl group.

FIG. 5 shows an exemplary scheme of the reactions of thermal (reaction a) and chemical treatment processes (reaction b) to form treatment-soluble derivatized sulfur-containing compounds. “Aromatic” refers to an aryl or heteroaryl ring; “H” refers to hydrogen; “Ar” refers to an aryl or heteroaryl ring; and “Alk” refers to an alkyl group.

FIG. 6 shows an exemplary set-up for the disclosed method of contacting the gas sample or liquefied petroleum gas sample including sulfur species with a derivatization agent to form derivatized sulfur species.

### DETAILED DESCRIPTION

The present disclosure relates to methods of determining the sulfur-content in a gas sample or liquefied petroleum gas sample including sulfur species. In some embodiments, the method includes derivatizing the sulfur-containing compounds and analyzing the derivatized sulfur-containing compounds. In some embodiments, the method determines the total concentration of the sulfur-containing compounds in the gas sample or liquefied petroleum gas sample.

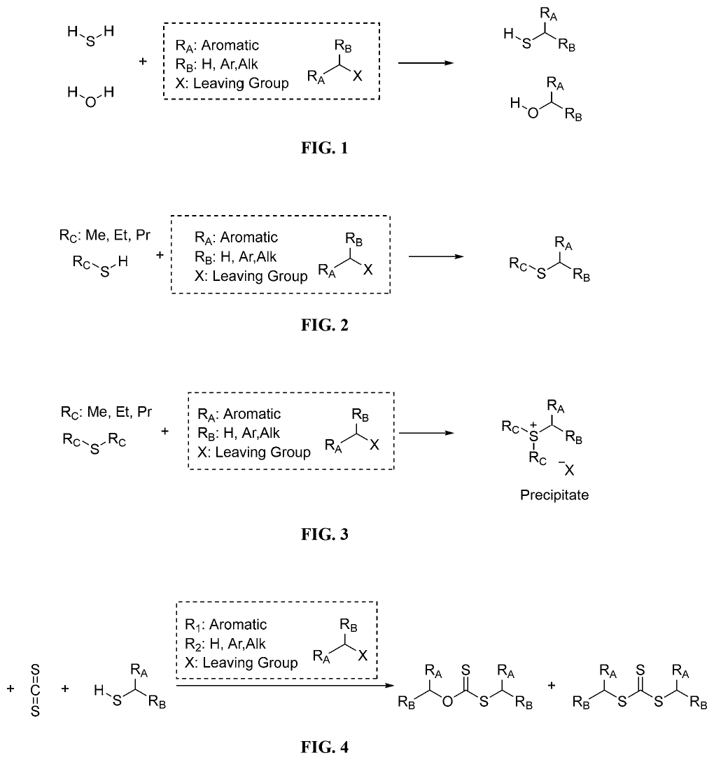

The use of a derivatization agent, such as a benzyl halide, may provide advantages over conventional methods of characterizing the sulfur-containing compounds in gas or liquefied petroleum gas samples. First, many derivatized sulfur-containing compounds, such as those containing a benzyl group, are UV-active compounds, which may be detected by a number of analytical techniques. Additionally, many derivatized sulfur-containing compounds, such as those containing a benzyl group, are solids or liquids, which are easier and safer to handle than gas samples. Furthermore, the disclosed derivatization agents may react with the types of sulfur-containing compounds commonly found in gas or liquefied petroleum gas samples, such as hydrogen sulfide, carbonyl sulfide, carbon disulfide, mercaptans, and dialkyl sulfides (FIGS. 1-4). In contrast, conventional methods of characterizing sulfur-containing compounds are only able to detect mercaptans, and not hydrogen sulfide, carbonyl sulfide, carbon disulfide, and dialkyl sulfides.

The term “alkyl” employed alone or in combination with other terms, refers to a saturated hydrocarbon group that may be straight-chained or branched. The term “Cn-m alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cn-m alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cn-m alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkoxy”, employed alone or in combination with other terms, refers to a group of formula-O-alkyl, where the alkyl group is as defined above. The term “Cn-m alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “carbonyl”, employed alone or in combination with other terms, refers to a —C(═O)— group, which also may be written as C (O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, Br, or I. In some embodiments, halo groups are F. In some embodiments, halo groups are Cl. In some embodiments, halo groups are Br. In some embodiments, halo groups are I.

The term “tosyl” refers to a toluenesulfonyl group (abbreviated Ts or Tos), a univalent functional group with the chemical formula —SO₂—C₆H₄—CH₃.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cn-m aryl” refers to an aryl group having from n to m ring carbon atoms. Aryl groups include, e.g., phenyl, naphthyl, indanyl, indenyl and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl. In some embodiments, the aryl group is naphthyl.

The term “heteroatom” used herein is meant to include boron, phosphorus, sulfur, oxygen and nitrogen.

The term “heteroaryl” or “heteroaromatic,” employed alone or in combination with other terms, refers to a monocyclic or polycyclic aromatic heterocycle having at least one heteroatom ring member selected from boron, phosphorus, sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-14, or 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, and the like.

The term “cycloalkyl,” employed alone or in combination with other terms, refers to a non-aromatic hydrocarbon ring system (monocyclic, bicyclic or polycyclic), including cyclized alkyl and alkenyl groups. The term “Cn-m cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6, 7, 8, 9, 10, 11, 12, 13, or 14 ring-forming carbons (C3-14). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C3-6 monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, bicyclo[2.2.1]heptanyl, bicyclo[2.2.2]octanyl and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from boron, nitrogen, sulfur, oxygen and phosphorus, and which has 4-14 ring members, 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic or polycyclic (e.g., having two or three fused or bridged rings) ring systems or spirocycles. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, 3-oxa-9-azaspiro[5.5]undecanyl, 1-oxa-8-azaspiro[4.5]decanyl, piperidinyl, morpholino, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 4,5,6,7-tetrahydrothiazolo[5,4-c]pyridinyl, and thiomorpholino.

The term “mercaptan,” used interchangeably with “thiol,” refers to any organosulfur compound of the form R—SH, where R represents an alkyl or other organic substituent.

The term “benzyl” refers to a substituent or molecular fragment possessing the structure R—CH₂—C₆H₅. Benzyl features a benzene ring (C₆H₆) attached to a methylene group (—CH₂—) group.

In some embodiments of the method of the present disclosure, the gas sample or liquefied petroleum gas sample is a gas sample. In some embodiments, the gas sample or liquefied petroleum gas sample is a gas sample. In some embodiments, the gas sample or liquefied petroleum gas sample is a liquefied petroleum gas sample.

In some embodiments, the gas sample or liquefied petroleum gas sample includes one or more sulfur-containing compounds. In some embodiments, the sulfur-containing compounds are selected from hydrogen sulfide, carbonyl sulfide, carbon disulfide, mercaptans, dialkyl sulfides, and combinations thereof.

In some embodiments, the gas sample or liquefied petroleum gas sample includes hydrogen sulfide, carbonyl sulfide, carbon disulfide, mercaptans, and dialkyl sulfides. In some embodiments, the gas sample or liquefied petroleum gas sample includes hydrogen sulfide. In some embodiments, the gas sample or liquefied petroleum gas sample includes carbonyl sulfide. In some embodiments, the gas sample or liquefied petroleum gas sample includes carbon disulfide. In some embodiments, the gas sample or liquefied petroleum gas sample includes mercaptans. In some embodiments, the gas sample or liquefied petroleum gas sample includes dialkyl sulfides.

In some embodiments, the gas sample or liquefied petroleum gas sample includes methanethiol. In some embodiments, the gas sample or liquefied petroleum gas sample includes ethanethiol. In some embodiments, the gas sample or liquefied petroleum gas sample includes propanethiol. In some embodiments, the gas sample or liquefied petroleum gas sample includes butanethiol. In some embodiments, the gas sample or liquefied petroleum gas sample includes pentanethiol. In some embodiments, the gas sample or liquefied petroleum gas sample includes hexanethiol. In some embodiments, the gas sample or liquefied petroleum gas sample includes heptanethiol. In some embodiments, the gas sample or liquefied petroleum gas sample includes octanethiol.

In some embodiments, the gas sample or liquefied petroleum gas sample includes dimethyl sulfide. In some embodiments, the gas sample or liquefied petroleum gas sample includes diethyl sulfide. In some embodiments, the gas sample or liquefied petroleum gas sample includes dipropyl sulfide. In some embodiments, the gas sample or liquefied petroleum gas sample includes dibutyl sulfide. In some embodiments, the gas sample or liquefied petroleum gas sample includes dipentyl sulfide. In some embodiments, the gas sample or liquefied petroleum gas sample includes dihexyl sulfide. In some embodiments, the gas sample or liquefied petroleum gas sample includes diheptyl sulfide. In some embodiments, the gas sample or liquefied petroleum gas sample includes dioctyl sulfide. In some embodiments, the gas sample or liquefied petroleum gas sample includes ethyl methyl sulfide. In some embodiments, the gas sample or liquefied petroleum gas sample includes propyl methyl sulfide. In some embodiments, the gas sample or liquefied petroleum gas sample includes butyl methyl sulfide. In some embodiments, the gas sample or liquefied petroleum gas sample includes propyl ethyl sulfide. In some embodiments, the gas sample or liquefied petroleum gas sample includes butyl ethyl sulfide. In some embodiments, the gas sample or liquefied petroleum gas sample includes propyl butyl sulfide.

In some embodiments, the derivatization agent is of formula (I):

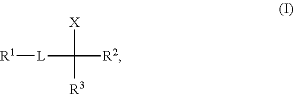

where:
X is halo or tosyl;
L is selected from bond, C₁₋₁₂ alkyl, C₂₋₁₂ alkenyl, C₂₋₁₂ alkynyl, and —C(O)—, where the C₁₋₁₂ alkyl, C₂₋₆ alkenyl, and C₂₋₆ alkynyl are optionally substituted with one or more substituents independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
R¹ is selected from C₆₋₁₀ aryl and 5-14 membered heteroaryl, where the C₆₋₁₀ aryl or 5-14 membered heteroaryl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, where the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b}, and
each R^{b} is independently selected from C₁₋₁₂ alkyl, C₂₋₁₂ alkenyl, and C₂₋₁₂ alkynyl.

In some embodiments, the derivatization agent is of formula (II):

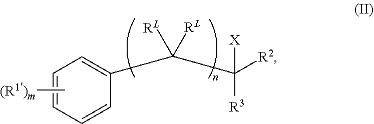

where:
X is halo or tosyl;
each R^{L} is independently selected from H, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
each R¹ is independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, where the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and C(O)R^{a};
R³ is selected from H and halo;
each R^{a} is independently selected from —OR^{b} and —NR^{b}R^{b},
each R^{b} is independently selected from C₁₋₁₂ alkyl, C₂₋₁₂ alkenyl, and C₂₋₁₂ alkynyl;
m is an integer selected from 0 to 5; and
n is an integer selected from 0 to 12.

In some embodiments, the derivatization agent is of formula (III-A) or (III-B):

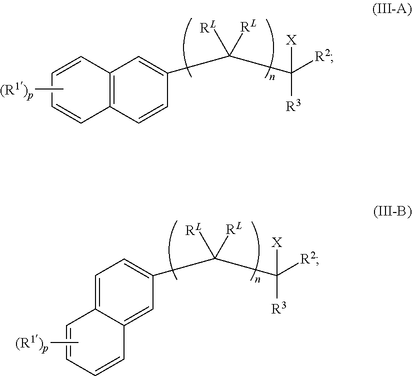

where:
X is halo or tosyl;
each R^{L} is independently selected from H, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
each R^{1′} is independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, where the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl;
n is an integer selected from 0 to 12; and
p is an integer selected from 0 to 6.

In some embodiments, the derivatization agent is of formula (III-A). In some embodiments, the derivatization agent is of formula (III-B).

In some embodiments, X is halo. In some embodiments, X is chloro. In some embodiments, X is X is bromo. In some embodiments, X is iodo. In some embodiments, X is tosyl.

In some embodiments, L is bond. In some embodiments, L is C₁₋₁₂ alkyl. In some embodiments, L is C₁₋₁₂ alkyl optionally substituted with one or more substituents independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a}.

In some embodiments, R¹ is C₆₋₁₀ aryl. In some embodiments, R¹ is C₆₋₁₀ aryl optionally substituted with one or more substituents independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a}. In some embodiments, R¹ is 5-14 membered heteroaryl. In some embodiments, R¹ is 5-14 membered heteroaryl optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a}.

In some embodiments, R¹ is phenyl. In some embodiments, R¹ is phenyl optionally substituted with one or more substituents independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a}. In some embodiments, R¹ is naphthyl. In some embodiments, R¹ is naphthyl optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a}.

In some embodiments, R² is H. In some embodiments, R² is halo. In some embodiments, R² is C₁₋₁₂ alkyl.

In some embodiments, R³ is H. In some embodiments, R³ is halo.

In some embodiments, each R^{L} is H.

In some embodiments, at least one R^{a} is —OR^{b}. In some embodiments, at least one R^{a} is —NR^{b}R^{b}.

In some embodiments, each R^{a} is —OR^{b}. In some embodiments, each R^{a} is —NR^{b}R^{b}.

In some embodiments, at least one R^{b} is C₁₋₁₂ alkyl. In some embodiments, at least one R^{b} is C₂₋₁₂ alkenyl. In some embodiments, at least one R^{b} is C₂₋₁₂ alkynyl.

In some embodiments, each R^{b} is C₁₋₁₂ alkyl. In some embodiments, each R^{b} is C₂₋₁₂ alkenyl. In some embodiments, each R^{b} is C₂₋₁₂ alkynyl.

In some embodiments, m is 0. In some embodiments, m is 1. In some embodiments, m is 2. In some embodiments, m is 3. In some embodiments, m is 4. In some embodiments, m is 5.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12.

In some embodiments, p is 0. In some embodiments, p is 1. In some embodiments, p is 2. In some embodiments, p is 3. In some embodiments, p is 4. In some embodiments, p is 5. In some embodiments, p is 5.

In some embodiments, the derivatization agent is selected from benzyl chloride, benzyl bromide, benzyl iodide, 1-(chloromethyl)naphthalene, 1-(bromomethyl)naphthalene, 1-(iodomethyl)naphthalene, 2-(chloromethyl)naphthalene, 2-(bromomethyl)naphthalene, and 2-(iodomethyl)naphthalene.

In some embodiments, the derivatization agent is benzyl chloride. In some embodiments, the derivatization agent is benzyl bromide. In some embodiments, the derivatization agent is benzyl iodide. In some embodiments, the derivatization agent is 1-(chloromethyl)naphthalene. In some embodiments, the derivatization agent is 1-(bromomethyl)naphthalene. In some embodiments, the derivatization agent is 1 (iodomethyl)naphthalene. In some embodiments, the derivatization agent is 2-(chloromethyl)naphthalene. In some embodiments, the derivatization agent is 2-(bromomethyl)naphthalene. In some embodiments, the derivatization agent is 2-(iodomethyl)naphthalene.

In some embodiments, the derivatization agent is dissolved in a solvent. In some embodiments, the solvent is an organic solvent.

In some embodiments, the solvent is a polar aprotic solvent. In some embodiments, the solvent is a polar protic solvent.

In some embodiments, the solvent is selected from acetonitrile, dichloromethane, ethyl acetate, tetrahydrofuran, 1,2-dichloroethane, benzene, toluene, and combinations thereof. In some embodiments, the solvent is dichloromethane. In some embodiments, the solvent is selected from tetrahydrofuran. In some embodiments, the solvent is 1,2-dichloroethane. In some embodiments, the solvent is toluene.

In some embodiments, contacting the gas sample or liquefied petroleum gas sample including one or more sulfur-containing compounds with a derivatization agent to form one or more derivatized sulfur-containing compounds occurs in the presence of a base.

In some embodiments, the base is selected from potassium carbonate, sodium carbonate, pyridine, and combinations thereof. In some embodiments, the base is potassium carbonate. In some embodiments, the base is sodium carbonate. In some embodiments, the base is pyridine.

In some embodiments, contacting the gas sample or liquefied petroleum gas sample including one or more sulfur-containing compounds with a derivatization agent to form one or more derivatized sulfur-containing compounds occurs at room temperature. In some embodiments, contacting the gas sample or liquefied petroleum gas sample including one or more sulfur-containing compounds with a derivatization agent to form one or more derivatized sulfur-containing compounds occurs at about 20° C. In some embodiments, contacting the gas sample or liquefied petroleum gas sample including one or more sulfur-containing compounds with a derivatization agent to form one or more derivatized sulfur-containing compounds occurs at about 25° C. In some embodiments, contacting the gas sample or liquefied petroleum gas sample including one or more sulfur-containing compounds with a derivatization agent to form one or more derivatized sulfur-containing compounds occurs at about 30° C. In some embodiments, contacting the gas sample or liquefied petroleum gas sample including one or more sulfur-containing compounds with a derivatization agent to form one or more derivatized sulfur-containing compounds occurs at about 40° C. In some embodiments, contacting the gas sample or liquefied petroleum gas sample including one or more sulfur-containing compounds with a derivatization agent to form one or more derivatized sulfur-containing compounds occurs at about 50° C. In some embodiments, contacting the gas sample or liquefied petroleum gas sample including one or more sulfur-containing compounds with a derivatization agent to form one or more derivatized sulfur-containing compounds occurs at a temperature greater than about 50° C.

In some embodiments, contacting the gas sample or liquefied petroleum gas sample including one or more sulfur-containing compounds with a derivatization agent to form one or more derivatized sulfur-containing compounds occurs at a temperature from about 0° C. to about 50° C., from about 10° C. to about 40° C., from about 20° C. to about 30° C., from about 0° C. to about 30° C., from about 10° C. to about 30° C., from about 20° C. to about 50° C., or from about 20° C. to about 40° C.

In some embodiments, one or more of the derivatized sulfur-containing compounds are soluble in the derivatization agent. In some embodiments, one or more of the derivatized sulfur-containing compounds are insoluble in the derivatization agent. In some embodiments, one or more of the derivatized sulfur-containing compounds are salts.

In some embodiments, analyzing the gas sample or liquefied petroleum gas sample includes a first analysis step, where the first analysis step includes analyzing the one or more derivatized sulfur-containing compounds that are soluble in the derivatization agent.

In some embodiments, analyzing the gas sample or liquefied petroleum gas sample includes a first analysis step, where the second analysis step includes treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.

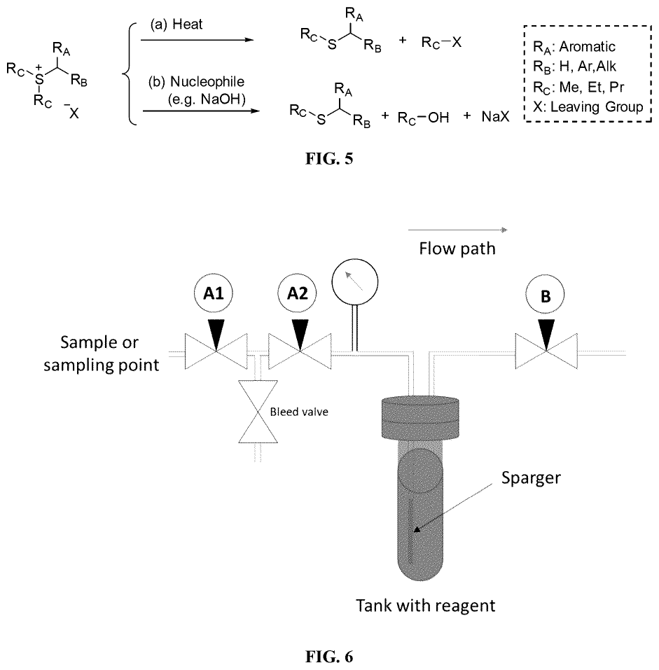

In some embodiments, treating the one or more derivatized sulfur species that are insoluble in the derivatization agent is a thermal treatment process. In some embodiments, treating the one or more derivatized sulfur species that are insoluble in the derivatization agent is a chemical treatment process. FIG. 5 shows exemplary reaction schemes for the thermal and chemical treatment processes.

In some embodiments, treating the one or more derivatized sulfur species that are insoluble in the derivatization agent are sulfonium salts.

In some embodiments, the chemical treatment process includes heating the sulfonium salt in the presence of a nucleophile to form a treatment-soluble derivatized sulfur-containing compound.

In some embodiments, the nucleophile is selected from hydroxide, an alkoxide, chloride, bromide, iodide, fluoride, thiol, a sulfide, and ammonia. In some embodiments, the nucleophile is hydroxide.

In some embodiments, the thermal treatment process includes heating the sulfonium salt in the presence of a nucleophile to form a treatment-soluble derivatized sulfur-containing compound.

In some embodiments, the derivatization agent does not react with hydrocarbons present in the gas sample or liquefied petroleum gas sample, where the hydrocarbons are selected from methane, ethane, propane, butane, pentane, hexane, heptane, octane, benzene, toluene, xylenes, and combinations thereof.

In some aspects, the present disclosure relates to methods of determining the sulfur-content in a gas sample or liquefied petroleum gas sample containing one or more sulfur-containing compounds, including:
contacting the gas sample or liquefied petroleum gas sample including one or more sulfur-containing compounds with a derivatization agent to form one or more derivatized sulfur-containing compounds;
performing a first analysis step including analyzing the one or more derivatized sulfur-containing compounds that are soluble in the derivatization agent; and
performing a second analysis step including treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.

In some embodiments, analyzing the sample includes gas chromatography (GC) analysis, gas chromatography-mass spectrometry (GC-MS) analysis, gas chromatography-infrared spectroscopy (GC-IR) analysis, gas chromatography-flame ionization detection (GC-FID) analysis, gas chromatography-vacuum ultraviolet (GC-VUV) spectroscopy analysis, liquid chromatography (LC) analysis, liquid chromatography-mass spectrometry (LC-MS) analysis, liquid chromatography-infrared spectroscopy (LC-IR) analysis, liquid chromatography-nuclear magnetic resonance (LC-NMR) analysis, capillary electrophoresis-mass spectrometry (CE-MS) analysis, high-performance liquid chromatography (HPLC) analysis, high-performance liquid chromatography diode array (HPLC-DAD) analysis, and/or high-performance liquid chromatography-mass spectrometry (HPLC-MS) analysis.

In some embodiments, analyzing the sample includes gas chromatography-flame ionization detection (GC-FID) analysis, gas chromatography-vacuum ultraviolet (GC-VUV) spectroscopy analysis, and/or high-performance liquid chromatography diode array (HPLC-DAD) analysis. In some embodiments, analyzing the sample includes gas chromatography-flame ionization detection (GC-FID) analysis. In some embodiments, analyzing the sample includes gas chromatography-vacuum ultraviolet (GC-VUV) spectroscopy analysis. In some embodiments, analyzing the sample includes high-performance liquid chromatography diode array (HPLC-DAD) analysis.

In some embodiments, the method determines the identity of the sulfur-containing compounds present in the gas sample or liquefied petroleum gas sample.

In some embodiments, the method determines the concentration of each of the sulfur-containing compounds present in the gas sample or liquefied petroleum gas sample.

### EXAMPLES

### Example 1. Method of Determining the Sulfur-Content in a Gas Sample or Liquefied Petroleum Gas Sample

A known volume of a gas sample or liquefied petroleum gas sample including sulfur species is passed through the set-up system shown in FIG. 5 to be contacted with a liquid derivatization agent dissolved in a solvent (e.g., dichloromethane) in the presence of a base (e.g., potassium carbonate). The sulfur species react quantitatively and are converted to stable, harmless derivatized sulfur species, some of which will remain in solution while some will precipitate as salts.

When sufficient gas or natural gas liquid sample has passed through the system, the system is isolated by closing the double block and bleed valve shown in FIG. 6. The quantity of gas or natural gas liquid passed through the reagent can be adjusted to meet the analytical requirements (e.g., limits of detection and quantification). The quantity of sample passing through the system is measured.

A first analysis of the species soluble in the reagent will be conducted. In line with the chemistry, the liquid phase will undergo a treatment (thermal or chemical) to dissolve salts and will be go through a second analysis to identify and quantify the sulfur species that formed salts.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining the sulfur-content in a gas sample or liquefied petroleum gas sample comprising one or more sulfur-containing compounds, the method comprising:
contacting the gas sample or liquefied petroleum gas sample comprising one or more sulfur-containing compounds with a derivatization agent to form one or more derivatized sulfur-containing compounds; and
analyzing the gas sample or liquefied petroleum gas sample comprising one or more derivatized sulfur-containing compounds to determine the sulfur-content;
wherein the method determines the total concentration of the one or more sulfur-containing compounds in the gas sample or liquefied petroleum gas sample, wherein the one or more sulfur-containing compounds are selected from hydrogen sulfide, carbonyl sulfide, carbon disulfide, mercaptans, dialkyl sulfides, and combinations thereof.

2. The method of claim 1, wherein the gas sample or liquefied petroleum gas sample is a gas sample.

3. The method of claim 1, wherein the gas sample or liquefied petroleum gas sample is a liquefied petroleum gas sample.

4. The method of claim 1, wherein the gas sample or liquefied petroleum gas sample comprises hydrogen sulfide, carbonyl sulfide, carbon disulfide, mercaptans, and dialkyl sulfides.

5. The method of claim 1, wherein the gas sample or liquefied petroleum gas sample comprises hydrogen sulfide.

6. The method of claim 1, wherein the gas sample or liquefied petroleum gas sample comprises dialkyl sulfides.

7. The method of claim 1, wherein the derivatization agent is of formula (I): 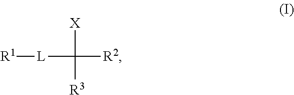
wherein:
X is halo or tosyl;
L is selected from bond, C₁₋₁₂ alkyl, C₂₋₁₂ alkenyl, C₂₋₁₂ alkynyl, and —C(O)—, wherein the C₁₋₁₂ alkyl, C₂₋₆ alkenyl, and C₂₋₆ alkynyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R¹ is selected from C₆₋₁₀ aryl and 5-14 membered heteroaryl, wherein the C₆₋₁₀ aryl or 5-14 membered heteroaryl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl.

8. The method of claim 1, wherein the derivatization agent is of formula (II): 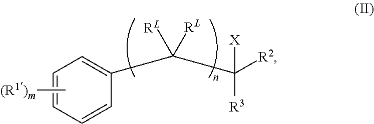
wherein:
X is halo or tosyl;
each R^{L} is independently selected from H, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
each R^{1′} is independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b},
each R^{b} is independently selected from C₁₋₁₂ alkyl, C₂₋₁₂ alkenyl, and C₂₋₁₂ alkynyl;
m is an integer selected from 0 to 5; and
n is an integer selected from 0 to 12.

9. The method of claim 1, wherein the derivatization agent is of formula (III-A) or (III-B): 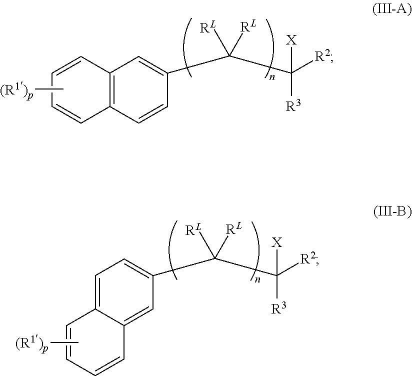
wherein:
X is halo or tosyl;
each R^{L} is independently selected from H, C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
each R^{1′} is independently selected from C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, 4-14 membered heterocycloalkyl, halo, and —C(O)R^{a};
R² is selected from H, halo, C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl, wherein the C₁₋₁₂ alkyl, C₂₋₁₂ alkenyl, C₂₋₁₂ alkynyl, C₆₋₁₀ aryl, C₃₋₁₄ cycloalkyl, 5-14 membered heteroaryl, and 4-14 membered heterocycloalkyl are optionally substituted with one or more substituents independently selected from C₁₋₁₂ alkyl, C₂₋₁₂ alkenyl, C₂₋₁₂ alkynyl, C₁₋₁₂ alkoxy, C₆₋₁₀ aryl, C₃₋₁₄ cycloalkyl, 5-14 membered heteroaryl, 4-14 membered heterocycloalkyl, halo, and —C(O)R^{a};
R³ is selected from H and halo;
each R^{a} is independently selected from —OR^{b} and —NR^{b}R^{b};
each R^{b} is independently selected from C₁₋₁₂ alkyl, C₂₋₁₂ alkenyl, and C₂₋₁₂ alkynyl;
n is an integer selected from 0 to 12; and
p is an integer selected from 0 to 6.

10. The method of claim 1, wherein the derivatization agent is selected from benzyl chloride, benzyl bromide, benzyl iodide, 1-(chloromethyl)naphthalene, 1-(bromomethyl)naphthalene, 1-(iodomethyl)naphthalene, 2-(chloromethyl)naphthalene, 2-(bromomethyl)naphthalene, and 2-(iodomethyl)naphthalene.

11. The method of claim 10, wherein the derivatization agent is benzyl bromide.

12. The method of claim 1, wherein contacting the gas sample or liquefied petroleum gas sample comprising one or more sulfur-containing compounds with a derivatization agent to form one or more derivatized sulfur-containing compounds occurs at room temperature.

13. The method of claim 1, wherein one or more of the derivatized sulfur-containing compounds are soluble in the derivatization agent.

14. The method of claim 1, wherein one or more of the derivatized sulfur-containing compounds are insoluble in the derivatization agent.

15. The method of claim 1, wherein one or more of the derivatized sulfur-containing compounds are salts.

16. The method of claim 1, wherein analyzing the gas sample or liquefied petroleum gas sample comprises a first analysis step, wherein:
the first analysis step comprises analyzing the one or more derivatized sulfur-containing compounds that are soluble in the derivatization agent.

17. The method of claim 16, wherein analyzing the gas sample or liquefied petroleum gas sample comprises a first analysis step, wherein:
the second analysis step comprises treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.

18. The method of claim 17, wherein treating the one or more derivatized sulfur species that are insoluble in the derivatization agent is a thermal treatment process or a chemical treatment process.

19. The method of claim 1, wherein the method determines the identity of the sulfur-containing compounds.

20. The method of claim 19, wherein the method determines the concentration of each of the sulfur-containing compounds.

21. The method of claim 1, wherein the derivatization agent does not react with hydrocarbons present in the gas sample or liquefied petroleum gas sample, wherein the hydrocarbons are selected from methane, ethane, propane, butane, pentane, hexane, heptane, octane, benzene, toluene, xylenes, and combinations thereof.

22. A method of determining the sulfur-content in a gas sample or liquefied petroleum gas sample comprising one or more sulfur-containing compounds, the method comprising:
contacting the gas sample or liquefied petroleum gas sample comprising one or more sulfur-containing compounds with a derivatization agent to form one or more derivatized sulfur-containing compounds;
performing a first analysis step comprising analyzing the one or more derivatized sulfur-containing compounds that are soluble in the derivatization agent; and
performing a second analysis step comprising treating the one or more derivatized sulfur-containing compounds that are insoluble in the derivatization agent to form one or more treatment-soluble derivatized sulfur-containing compounds and analyzing the one or more treatment-soluble derivatized sulfur-containing compounds.
